(19) United States
(12) Patent Application Publication
(10) Pub. No.: US 2004/0029783 A1
(43) Pub. Date: Feb. 12, 2004

(54) Use of 2,3 alkylcarbonyloxybenzoic acids in the treatment of anthrax

(76) Inventor: Karen J. Stec, Barrington, IL (US)

Correspondence Address: Jeffrey M. Hoster, 13 Woodland Drive, Lemont, IL, 60439, US

(21) Appl. No.: 10/284,768

(22) Filed: Oct. 31, 2002

Related U.S. Application Data
(60) Provisional application No. 60/336,006, filed on Nov. 2, 2001.

(51) Int. Cl. A61K 38/14; A61K 31/704; A61K 31/545; A61K 31/496; A61K 31/225; A61K 31/65

(52) U.S. Cl. 514/8; 514/548; 514/29; 514/152; 514/200; 514/252.13; 514/36; 514/192

(57) ABSTRACT

A method for treating inhalation anthrax is disclosed. The inventive method comprises the use of 2,3-alkylcarbonyloxybenzoic acid and salts thereof in the prevention and treatment of lung damage caused by Bacillus anthracis and toxins produced by the bacterium. The 2,3-alkylcarbonyloxybenzoic acid may be used alone or in combination with other therapeutic agents such as antibiotics.

### FIELD OF THE INVENTION

This invention is directed to the field of treating anthrax. More particularly, this invention is directed to the field of repairing damage to tissue such as lung tissue caused by the Bacillus anthracis bacterium.

### BACKGROUND OF THE INVENTION

Inhalation anthrax is a disease resulting from inhalation of spores of the bacterium Bacillus anthracis. The spores are the dormant form of the bacterium. However, upon germination in the lungs of human or animal subjects, the spores become active. Very rapidly following germination, the bacteria multiply and produce toxins. These toxins cause inflammation, fluid buildup and hemorrhaging in the lungs, followed in most cases by rapid death.

There have been studies of the manifestations of anthrax in the lungs. One such study was published in the Proc. Natl. Acad. Sci. USA, Vol. 90, pp. 2291-2294, March, 1993 by Faina A. Abramova et al (“Abramova”). In this study, autopsies of 42 subjects who died of inhalation anthrax were conducted. All of the subjects were found to have hemorrhagic necrosis of the thoracic lymph nodes in the lymphatic drainage of the lungs and hemorrhagic mediastinitis. The lungs of the subjects also evidenced edema and increased vascular permeability. The anthrax toxins were considered to be the cause of the vascular damage, resulting in the fluid buildup and hemorrhaging. The toxins were found to have three components: protective antigen, edema factor, and lethal factor. Generally, the mechanism of anthrax in the lungs appear to be similar to manifestations of sepsis.

Certain antibiotics are known to prevent inhalation anthrax, but only if the antibiotics are administered prior to spore germination in the lungs. There presently are no known therapeutic agents for the treatment for inhalation anthrax once lung activity has begun. This is especially problematic in that early symptoms of inhalation anthrax resemble flu symptoms. When anthrax enters the secondary stage, death of the subjects is near certain, generally resulting from the vascular permeability and resulting pulmonary edemas caused by the toxins. Even if antibiotics are used effectively in treatment, there has been no method reported for reversing the above-described lung damage.

Thus, there is a need for a therapeutic agent for the treatment of inhalation anthrax. The present invention is directed to such a therapeutic agent.

### SUMMARY OF THE INVENTION

The invention encompasses methods for treating inhalation anthrax by the use of 2,3-alkylcarbonyloxybenzoic acids in which the alkylcarbonyloxy group has 2-18 carbon atoms. One particularly preferred compound is 2,3-diacetoxybenzoic acid. The preferred method of treatment involves administering 2,3-alkylcarbonyloxybenzoic acid to a subject known or suspected to have inhalation anthrax. The 2,3-alkylcarbonyloxybenzoic acid can be administered by any known therapeutic method, and in combination with other compounds known or believed to prevent or treat anthrax. More than one of the 2,3-alkylcarbonyloxybenzoic acid compounds can also be blended.

### DETAILED DESCRIPTION OF THE PREFERRED EMBODIMENTS

In a preferred embodiment, a therapeutically effective amount of a compound selected from the group consisting of 2,3-alkylcarbonyloxybenzoic acids and salts thereof in which the alkylcarbonyloxy group has 2-18 carbon atoms is administered to subjects who have indications for inhalation anthrax. (For purposes of this application, any reference to 2,3-alkylcarbonyloxybenzoic acids shall be construed to include the salts thereof.) The 2,3-alkylcarbonyloxybenzoic acids have been shown to ameliorate the damage resulting from increased vascular permeability and the resulting pulmonary edema caused by increased microvascular permeability to protein and solutes resulting from Adult Respiratory Distress Syndrome and sepsis. This effect is detailed in U.S. Pat. No. 5,504,111, issued Apr. 2, 1996 to Flavin et al.

A particularly preferred compound in the class of 2,3-alkylcarbonyloxybenzoic acid compounds is 2,3-diacetoxybenzoic acid. In an especially preferred embodiment, 2,3-diacetoxybenzoic acid is administered to a subject, either via intravenous or aerosol spray means in a therapeutically effective amount. This amount is preferably, but not exclusively, in the range of 1-100 mg/kg of body weight of the subject. More preferably, the range is 5-50 mg/kg of body weight, and most preferably, the range is 5-20 mg/kg of body weight. While preferred, these ranges are exemplary only. Combinations with other therapeutic agents, the delivery system, and the stage of the anthrax disease may result in use of amounts outside the above-described ranges. The dosages described above can be administered over the period of time necessary to show effectiveness. While not wishing to be bound by theory, it is believed that, following administration to a subject, at least a percentage of 2,3-diacetoxybenzoic acid is converted to 2,3-dihydroxybenzoic acid, which has therapeutic effectiveness. Thus, all or a portion of an anthrax treatment product may also comprise 2,3-dihydroxybenzoic acid, whether administered directly or through chemical conversion in the subject.

The 2,3-alkylcarbonyloxybenzoic acid can be used either on a preventative basis, or after anthrax disease has been diagnosed, either in the initial or secondary stage of diagnosis. The 2,3-alkylcarbonyloxybenzoic acid can be administered to human or animal subjects.

The 2,3-alkylcarbonyloxybenzoic acid is typically produced in solid form. In preparing the therapeutic product, it is preferably formulated into a liquid form by use of a sodium bicarbonate buffer present in an amount to effectively solubilize and stabilize the acid. The acid is also converted to a sodium salt. The buffered salt can then be constituted into aerosol form using conventional methods of forming aerosol products. Administration of the aerosol form can be accomplished via either nasally or orally, including forms such as metered inhaled forms.

Treatment with 2,3-alkylcarbonyloxybenzoic acid can be used as a sole treatment mechanism, or in combination with other therapeutic agents. For example, 2,3-alkylcarbonyloxybenzoic acid can be administered in conjunction with antibiotic therapy. Antiobiotics have been shown to treat the Bacillus anthracis bacterium, but not the damage caused by the toxins produced by the bacteria. Thus, a combination of 2,3-alkylcarbonyloxybenzoic acid and one or more antibiotics is expected to be an especially useful therapeutic combination. The antibiotics may be selected from the group comprising ciprofloxacin, doxycycline, rifampin, vancomycin, imipenem, chloramphenicol, penicillin, clindamycin, clarithromycin, and analogs, homologs, and derivatives thereof which have antibiotic functionality.

2,3-alkylcarbonyloxybenzoic acid can also be combined with other therapeutic agents which are used in the treatment of sepsis or other conditions in which bacteria and their toxins spread through lung tissues. An example of such a therapeutic agent is drotrecogin alfa (Xigris ® brand, Eli Lilly & Co.), but any similar therapeutic agents also can be used. A benefit of co-treatment with drotrecogin alfa is that the mechanism of drotrecogin alfa differs from that of 2,3-alkylcarbonyloxybenzoic acid.

Thus, the present invention teaches a novel method for the treatment of inhalation anthrax, and the novel use of 2,3-alkylcarbonyloxybenzoic acids and salts thereof.

What is claimed is:

1. A method for treating inhalation anthrax comprising administering to a human or animal subject an effective therapeutic amount of one or more compounds selected from the group consisting of 2,3-alkylcarbonyloxybenzoic acids and salts thereof wherein the alkylcarbonyl group has 2-18 carbon atoms.

2. The method of claim 1 wherein said 2,3-alkylcarbonyloxybenzoic acid is 2,3-diacetoxybenzoic acid.

3. The method of claim 1 wherein said 2,3-alkylcarbonyloxybenzoic acid is formulated in liquid form in the presence of a buffer.

4. The method of claim 3 wherein said buffer is a sodium bicarbonate buffer such that a sodium salt of said 2,3-alkylcarbonyloxybenzoic acid is formed.

5. The method of claim 1 wherein said 2,3-alkylcarbonyloxybenzoic acid is administered to said subject in aerosol spray form.

6. The method of claim 5 wherein said 2,3-alkylcarbonyloxybenzoic acid is administered via nasal or mouth passages.

7. The method of claim 1 wherein said 2,3-alkylcarbonyloxybenzoic acid is administered to said subject in liquid form via intravenous means.

8. The method of claim 1 wherein said 2,3-alkylcarbonyloxybenzoic acid is administered to said subject together with an antibiotic.

9. The method of claim 8 wherein said antibiotic is selected from the group comprising ciprofloxacin, doxycycline, rifampin, vancomycin, imipenem, chloramphenicol, penicillin, clindamycin, clarithromycin, and analogs, homologs, and derivatives thereof which have antibiotic functionality.

10. The method of claim 1 wherein said 2,3-alkylcarbonyloxybenzoic acid is administered to said subject together with at least one therapeutic agent for the treatment of sepsis.

11. The method of claim 10 wherein said therapeutic agent for the treatment of sepsis is drotrecogin alfa.

12. The method of claim 1 wherein said 2,3-alkylcarbonyloxybenzoic acid is administered in an amount of from about 1 mg. to about 100 mg. per kg. of body weight of said subject.

13. The method of claim 12 wherein said 2,3-alkylcarbonyloxybenzoic acid is administered in an amount of from about 5 mg. to about 50 mg. per kg. of body weight of said subject.

14. The method of claim 13 wherein said 2,3-alkylcarbonyloxybenzoic acid is administered in an amount of from about 5 mg. to about 20 mg. per kg. of body weight of said subject.

15. The use in an effective therapeutic amount of one or more compounds selected from the group consisting of 2,3-alkylcarbonyloxybenzoic acids and salts thereof in which the alkylcarbonyl group has 2-18 carbon atoms in the treatment of inhalation anthrax.

16. The use of claim 15 wherein said 2,3-alkylcarbonyloxybenzoic acid is 2,3-diacetoxybenzoic acid.

17. The use of claim 15 wherein said effective therapeutic amount is an amount of from about 1 mg. to about 100 mg. per kg. of body weight of said subject.

18. The use of claim 17 wherein said effective therapeutic amount is an amount of from about 5 mg. to about 50 mg. per kg. of body weight of said subject.

19. The use of claim 18 wherein said effective therapeutic amount is an amount of from about 5 mg. to about 20 mg. per kg. of body weight of said subject.

20. The use in an effective therapeutic amount of one or more compounds selected from the group consisting of 2,3-alkylcarbonyloxybenzoic acids and salts thereof in which the alkylcarbonyl group has 2-18 carbon atoms combined with an effective therapeutic amount of one or more antibiotics in the treatment of inhalation anthrax.

21. The use of claim 20 wherein said antibiotics are selected from the group comprising ciprofloxacin, doxycycline, rifampin, vancomycin, imipenem, chloramphenicol, penicillin, clindamycin, clarithromycin, and analogs, homologs, and derivatives thereof which have antibiotic functionality.

22. The use of claim 15 in an effective therapeutic amount of one or more compounds selected from the group consisting of 2,3-alkylcarbonyloxybenzoic acids and salts thereof in which the alkylcarbonyl group has 2-18 carbon atoms combined with an effective therapeutic amount of at least one therapeutic agent for the treatment of sepsis.

23. The use of claim 22 wherein said therapeutic agent for the treatment of sepsis is drotrecogin alfa.
